(19) United States
(12) Patent Application Publication
(10) Pub. No.: US 2011/0245148 A1
(43) Pub. Date: Oct. 6, 2011

(54) ACETIC ACID AND A BUFFER

(75) Inventors: Michael Christian Givskov, Humlebaek (DK); Thomas Bjarnsholt, Soborg (DK); Preben Homoe, Koge (DK); Niels Hoiby, Farum (DK); Peter Østrup Jensen, Copenhagen S (DK); Helle Krogh Johansen, Horsholm (DK); Klaus Kirketerp-Moller, Koge (DK)

(73) Assignees: DANMARKS TEKNISKE UNIVERSITET, Lyngby (DK); BISPEBJERG HOSPITAL, Copenhagen NV (DK); RIGSHOSPITALET COPENHAGEN UNIVERTISY HOSPITAL, Copenhagen O (DK)

(21) Appl. No.: 12/999,756

(22) PCT Filed: Jun. 23, 2009
(86) PCT No.: PCT/DK2009/050141

§ 371 Date: Jun. 17, 2011

Related U.S. Application Data
(60) Provisional application No. 61/074,804, filed on Jun. 23, 2008.

(30) Foreign Application Priority Data
Jun. 23, 2008 (EP) 08158755.2

(51) Int. Cl. A01N 37/18; A01N 37/02; A01N 43/34; A01N 43/04; A61K 31/19; A61P 31/00; A01P 1/00

(52) U.S. Cl. 514/2.3; 514/557; 514/210.02; 514/154; 514/203; 514/23

(57) ABSTRACT

The present invention relates to a composition comprising: a) 0.01-20% wt/wt acetic acid and b) a physiologically tolerable buffer capable of maintaining acetic acid at a pH in the range of 2-7; and use of such a composition as an antimicrobial agent.

### TECHNICAL FIELD OF THE INVENTION

The present invention relates to a composition comprising acetic acid and a buffer capable of maintaining the pH value of the acetic acid in the range of 2-7, wherein said buffer is physiologically tolerable. The composition of the present invention has been found to have an anti-microbial effect.

### BACKGROUND OF THE INVENTION

Microbes, in particular bacteria, are known to cause various types of infections in both humans and animals and also to cause problems in industrial equipment especially in cases were a high standard of hygiene is required. Antibiotics can be used to either kill or inhibit the growth of unwanted microbes and it is usually the choice of treatment for infections. However, the world wide increase in antibiotic resistant microbes has limited the effect of traditional treatments making it very difficult to treat infections that were once treatable. A particular problem is infections were the bacteria are capable of forming a so called biofilm as such infections typically tolerate the highest deliverable doses of antibiotics. Such infections develop commonly on inert surfaces of medical devices and implants, but they are also associated with cystic fibrosis, endocarditis, rhinosinusitis, chronic otitis media and chronic wounds. Due to this antibiotic resistance it is important to devise new treatment scenarios which efficiently enable eradication of unwanted microbes. Furthermore, in relation to infections in humans or animals it is imperative that the treatment is non-toxic to the hosts and physiologically acceptable.
Martineau L and Dosch HM (2007), Journal of Applied Microbiology, 103, 297-304, describes “Biofilm reduction by a new burn gel that targets nociception”.
Akiyama H et al (1999), Arch Dermatol Res, 291, 570-573, describes “Effects of acetic acid on biofilms formed by Staphylococcus aureus”.

### SUMMARY OF THE INVENTION

Thus, one aspect of the invention relates to a composition comprising:
a) 0.01-20% wt/wt acetic acid; and
b) a physiologically tolerable buffer capable of maintaining acetic acid at a pH in the range of 2-7

Another aspect of the present invention is the use of a composition comprising 0.01-20% wt/wt acetic acid as an antimicrobial agent administered to microbial biofilm.

Another aspect of the present invention relates to the use and methods of using a composition comprising acetic acid and buffer to inhibit microbial growth, such as for cleaning of industrial or medical equipment.

A final aspect of the present invention is a composition comprising: a) 0.01-20% wt/wt acetic acid; and b) a physiologically tolerable buffer capable of maintaining acetic acid at a pH in the range of 2-7 for use as a medicament.

### BRIEF DESCRIPTION OF THE FIGURES

FIG. 1 shows antimicrobial effect of 0.5% acetic acid on 24 hours old static P. aeruginosa biofilms grown in AB minimal medium adjusted to different pH values. AB minimal medium without acetic acid adjusted to different pH values was used as control. The biofilm bacteria were harvested and plated on LB plates in order to determine the CFU after treatment (For visual appearance “no growth” is arbitrarily given a CFU value of 2).

FIG. 2 demonstrates the antimicrobial effect of 0.5% acetic acid in synergy with increasing concentrations of tobramycin on 24 hour old static P. aeruginosa biofilms at pH 6.85. AB minimal medium supplemented with increasing concentrations of tobramycin served as control. The biofilm were harvested and plated on LB plates to determine the CFU after treatment (For visual appearance “no growth” is arbitrarily given a CFU value of 2).

FIG. 3 demonstrates the synergistic antimicrobial effect of 0.5% acetic acid at different pH values with increasing concentrations of tobramycin on 24 hour old static P. aeruginosa biofilms. AB minimal medium without acetic acid, but supplemented with increasing concentrations of tobramycin served as control. The biofilm bacteria were harvested and plated on LB plates in order to determine the CFU after treatment (For visual appearance “no growth” is arbitrarily given a CFU value of 2).

FIG. 4 demonstrates the synergistic, antimicrobial effect of 0.5% acetic acid at different pH values in combination with increasing concentrations of tobramycin on 24 hour old static biofilms on a clinical non-mucoid P. aeruginosa isolate. AB minimal media without acetic acid but supplemented with increasing concentrations of tobramycin served as control. The biofilm were harvested and plated on LB plates to determine the CFU after treatment (For visual appearance “no growth” is arbitrarily given a CFU value of 2).

FIG. 5 demonstrates the synergistic, antimicrobial effect of 0.5% acetic acid at different pH values in with increasing concentrations of tobramycin on 24 hour old static biofilms of a clinical mucoid P. aeruginosa isolate. AB minimal medium without acetic acid, but supplemented with increasing concentrations of tobramycin was used as control. The biofilm were harvested and plated on LB plates to determine the CFU after treatment (For visual appearance “no growth” is arbitrarily given a CFU value of 2).

FIG. 6 shows a chronic heel ulcer infected with P. aeruginosa in a 38 year old male patient with type 2 mellitus diabetes prior to treatment with buffered acetic acid solution (day 0). At this stage several well known antibacterial treatments had been tried.

FIG. 7 shows the heel ulcer of FIG. 6 after 11 days of treatment with buffered acetic acid solution (day 11). The mucoid infection has been eradicated and the ulcer is now healing normally.

FIG. 8 shows a chronic leg ulcer infected with P. aeruginosa in a 73 years old female patient with type 2 mellitus diabetes prior to treatment with buffered acetic acid solution (day 0). Cultures prior to treatment have shown Pseudomonas aeruginosa and Staphylococcus aureus. The patient received anti-Staphylococcus treatment due to infected toe on the contralatteral leg (Heracillin).

FIG. 9 shows the leg ulcer of FIG. 8 after 3 days of treatment with buffered acetic acid solution (day 3). The mucoid infection has been significantly reduced and the ulcer has attained a dark red color indicating reestablishment of the normal wound healing process.

FIG. 10 shows the leg ulcer of FIGS. 8 and 9 after 6 days of treatment with buffered acetic acid solution (day 6). The mucoid infection has been eradicated and the ulcer is now healing.

The present invention will now be described in more detail in the following.

### DETAILED DESCRIPTION OF THE INVENTION

### Composition

The present invention relates to a composition comprising:
a) 0.01-20% wt/wt acetic acid
b) a physiologically tolerable buffer capable of maintaining the acetic acid at a pH in the range of 2-7

The inventors of the present invention have found that acetic acid in the non-dissociated form as given by the formula CH₃COOH is capable of reducing microbial growth when it is present in its acidic form. In the context of the present invention the term “acidic form” used in relation to acetic acid means that it is present as CH₃COOH. As an equilibrium between CH₃COO⁻ and CH₃COOH will exist when the pH is at the pKa value 4.76, 50% of the total amount of CH₃COO⁻ and CH₃COOH is non-dissociated CH₃COOH at the pKa value.

As also shown in the examples, HCl present in the same buffer does not exert a similar effect on microbial growth as acetic acid even when the two compounds are used at the same pH. This indicates that it is not the acidic pH which reduces the microbial growth but the acetic acid molecule itself in its non-dissociated form. To maintain acetic acid in its active protonated form a buffer capable of maintaining acetic acid in its acidic form is included in the composition of the present invention.

In the context of the present invention the term “physiologically tolerable buffer” is to be understood as buffers used according to the invention resulting in solutions that are nontoxic to recipients at the dosages and concentrations employed and which are sterile, endotoxin-free and pyrogen-free. Sterility and toxicity may be assessed according to the official monographs of U.S. Pharmacopeia e.g. sterility test USP 71, bacterial endotoxins test USP 85 and pyrogen test USP 151. Also a physiologically tolerable buffer in the present context is non-carcinogenic and non-mutagenic in the applied dosages and concentrations.

The term buffer is well known as a general description of a solution containing either a weak acid and its salt or a weak base and its salt, which is resistant to changes in pH.

In the context of the present invention the term “buffer capable of maintaining acetic acid at a pH in the range of 2-7” is to be understood as a buffer which is capable of maintaining acetic acid in its acidic form also when the composition is added to e.g. a microbial biofilm, a wound or any other environment which is capable of affecting the pH of the composition. In this context the term “capable of maintaining” is to be understood as a buffer which is capable of maintaining the pH of the acetic acid in the specified interval for a period of more than 2 hours, such as more than 3 hours, or more than 4 hours, or more than 5 hours, or more than 6 hours, or more than 7 hours, or more than 8 hours, or more than 9 hours, or more than 10 hours, or more than 15 hours, or more than 20 hours, or more than 24 hours, or more than 36 hours, or more than 48 hours, or more than 72 hours, or more than 96 hours, or more than 100 hours, or more than 150 hours. If the composition of the present invention is added to a wound, such as a chronic wound the pH may be measured by pH indicator paper or stick, such as the commercially available Hydrion paper.

Examples of other weak acids which may be used in the composition of the present invention instead of acetic acid include but are not limited to: Aluminium diacetate, citric acid, methane acid, propane acid, butane acid, boric acid.

Different buffers are capable of maintaining acetic acid at a pH in the range of 2-7 while being physiologically tolerable and examples of these include but are not limited to: sodium acetate, sodium bicarbonate, phosphate buffers, tris buffers and HEPES buffer. Thus in a preferred embodiment the composition of the present invention may contain a buffer selected from this group. One advantage of using a buffer in the composition of the present invention is the capability of the buffer to maintain the pH at a given desired value for longer than the equivalent compositions with no buffer present. This is for example advantageous when applying the composition to patients since the composition is active for longer periods of time and the composition need not be re-applied or changed as often as when no buffer is present.

Another advantage of the composition of the present invention is that it is based on well known and inexpensive materials. Furthermore, the composition of the present invention is non-toxic; i.e. it is physiologically tolerable to humans (and animals) which makes it particularly suitable for treatment of infections in humans (and animals). Importantly, toxic and/or carcinogenic buffers such as, triethanolamine, tri- and di-ethylamine and salts thereof are not comprised in the present invention as the composition is administered in relatively large amounts, possibly to compromised or sensitive tissue such as wounds or lung tissue. Additionally increment of the pH using a buffer enables sustained oral usage without destroying the enamel of the teeth.

The composition of the present invention is sterile, i.e. it is essentially free of transmissible agents such as fungi, bacteria or viruses. A sterile composition may be achieved using sterile components and/or by sterilisation of the finished composition and/or by filtration through sterile filtration membranes, prior to being placed into a container having a sterile access port, for example, an intravenous solution bag or vial having a stopper pierceable by a hypodermic injection needle.

In one embodiment the buffer of the present invention may in particular be capable of maintaining pH of acetic acid in the range of 2-6.5, such as 2-6, or 2-5.5, or 2-5, or 2.5-7, or 2.5-6.5, or 2.5-6, or 2.5-5.5, or 2.5-5, or 3-7, or 3-6.5, or 3-6, or 3-5.5, or 3-5, or 3.5-7, or 3.5-6.5, or 3.5-6, or 3.5-5.5, or 3.5-5, or 4-7, or 4-6.5, or 4-6, or 4-5.5, or 4-5.

In another embodiment the composition of the present invention may comprise 0.01-15% wt/wt acetic acid, or 0.01-10% wt/wt acetic acid, or 0.01-5% wt/wt acetic acid, or 0.05-20% wt/wt acetic acid, 0.05-15% wt/wt acetic acid, 0.05-10% wt/wt acetic acid, or 0.01-5% wt/wt acetic acid, 0.10-20% wt/wt acetic acid, 0.10-15% wt/wt acetic acid, 0.1-10% wt/wt acetic acid, or 0.10-5% wt/wt acetic acid, or 0.5-20% wt/wt acetic acid, or 0.5-15% wt/wt acetic acid, or 0.5-10% wt/wt acetic acid, or 0.5-5% wt/wt acetic acid, or 1.0-20% wt/wt acetic acid, or 1.0-15% wt/wt acetic acid, or 1.0-10% wt/wt acetic acid, or 1.0-5% wt/wt acetic acid, or 2.5-20% wt/wt acetic acid, or 2.5-15% wt/wt acetic acid, or 2.5-10% wt/wt acetic acid, or 2.5-5% wt/wt acetic acid, or 5-20% wt/wt acetic acid, or 5-15% wt/wt acetic acid, or 5-10% wt/wt acetic acid, or 10-20% wt/wt acetic acid, or 10-15% wt/wt acetic acid.

Buffers are known to have different buffering capacities and the amount of buffer it is relevant to use therefore depends on among other things the choice of buffer and the conditions under which the composition is to be used, e.g. whether it is used to treat a medical wound, the lungs or to clean industrial or medical equipment.

The inventors of the present invention have shown that when pH of a composition of the present invention is 5 or above the presence of tobramycin in the composition is able to exert a synergistic, antimicrobial effect with respect to decreasing the amount of viable bacteria compared to the same composition without tobramycin. Hence it may be an advantage to include an antibiotic in the composition of the present invention especially if the pH of the composition is above 4.5, such as above 5.

Thus in one embodiment of the present invention the composition may further comprise an antibiotic. Examples of such suitable antibiotics include but are not limited to: amino glycosides, macrolides, fluoroquinolones, ceftazidimes, tetracyclines, sulfonamides, beta-lactams and antimicrobial peptides.

In another embodiment the composition of the present invention may further comprise one or more compounds selected from the group consisting of but not limited to: detergents, anti-microbial agents and disinfectants. The term “microbe” or “microbial” is in the context of the present invention to be understood as including both proteo-bacteria, archea, fungi and yeast.

The composition of the present invention may in one embodiment be a fluid, such as a liquid, gel, gaseous or aerosol composition. If the composition of the present invention is a liquid it may in particular be a solution, such as an aqueous solution. The composition may also be in a dry form, e.g. suspended or adsorbed in a solid and/or dry material. The dry material may preferably be a powder such as an adsorbent powder. Such dry compositions may be activated upon contact with fluids, such as bodily fluids, e.g. liquids in or around a wound.

Use of the Composition Comprising 0.01-20% wt/wt Acetic Acid as an Antimicrobial Agent Administered to Microbial Biofilm

In another aspect of the present invention the inventors found that the use of a composition comprising 0.01-20% wt/wt acetic acid, i.e. without buffer, as an antimicrobial agent administered to microbial biofilm, was effective. The inventors anticipate that when not applying buffer the composition is not as pH stable as the above described composition, but is nevertheless capable of eradicating biofilm forming bacteria. In a preferred embodiment the composition comprising 0.01-20% wt/wt acetic acid may be used to clean and/or disinfect industrial and medical equipment, especially equipment where a high hygiene standard is necessary. Examples of industries in which it is foreseen that a composition according to the present invention may be used include but are not limited: any food-producing industry, such as the dairy industry, the meat, poultry or aquaculture industry or the brewery industry. Examples of devices which may be treated with a composition of the present invention include but are not limited to medical or industrial devices, such as surgical instruments, catheters or pipelines. It should be noted that embodiments and features, described in the context of the composition comprising both acetic acid and a buffer may also apply to the above composition comprising only acetic acid and vice versa.

### Use of the Composition of the Present Invention Comprising Acetic Acid and a Buffer

The inventors of the present invention have found that acetic acid in its acidic form has an antimicrobial effect. Hence the present invention also relates to the use of a composition comprising: a) 0.01-20% wt/wt acetic acid; and b) a physiologically tolerable buffer capable of maintaining acetic acid at a pH in the range of 2-7 as an antimicrobial agent. The term “antimicrobial” is in the context of the present invention to be understood as a compound (or agent) which is capable of reducing the growth of a microbe; in particular said compound or agent may be able of killing a microbe.

The present invention also relates to a method of reducing bacterial growth comprising administration of a composition of the present invention to a microbe. The method may involve the steps of 1) identifying an area affected by bacterial growth, 2) applying the composition of the present inventions to said area, possibly using a spray, dressing, sponge, or by flushing the area, 3) repeating the application until the bacterial growth has been reduced, preferably eradicated.

Microbes may grow as individual organisms planktonic or proliferate into aggregates also known as microbial biofilms.

Biofilms are often found in chronic bacterial infections in or on humans and are known to be very difficult to eradicate with conventional antibiotics. The inventors of the present invention have found that acetic acid in its acidic form is very useful in treating such chronic bacterial infections and that it is the molecule itself which has this effect rather than the pH of the composition. Furthermore, when the composition of the present invention is combined with antibiotics a synergistic effect is achieved enabling effective treatment of otherwise chronic or persistent infections. Hence in a particular embodiment the composition of the present invention may be administered to an infection comprising a microbial biofilm. Unwanted microbial growth occurs under many different conditions, such as infections of humans, animals or plants, in industrial settings, e.g. pipelines in the oil industry, or those of food production or medical equipment were high hygiene is required. It is contemplated that the composition of the present invention may be used to reduce microbial growth and viability in all of these circumstances.

Examples of microbes which may be treated with a composition according to the present invention include but are not limited to: Bacteria, in particular proteobacteria, such as Pseudomonas aeruginosa, Burkholderia cepacia and 10 Escherichia coli and Gram positive bacteria such as Staphylococcus aureus and Staphylococcus epidermis.

Biofilm forming bacteria are often opportunistic and will cause chronic infections in mammals with a reduced immune response and/or in mammals with inserted foreign bodies such as hip, knee or heart valve replacements and internal and external catheters. In one embodiment of the present invention the composition may be applied to infections comprising a microbial biofilm in patients with a reduced immune response. The reduced immune response may have a variety of causes including cystic fibrosis, diabetes, age or obesity. Also administration of immunosuppressive medicine may cause a reduced immune response.

Examples of infections which the composition of the present invention may be used to treat include but are not limited to: infections where a biofilm is present, such as infections found in cystic fibrosis, chronic wounds, the lungs, chronic otitis media endocarditis, rhinosinusitis, chronic obstructive pulmonary disease (COPD), and on or around a catheter, prostethic device, implant or the oral cavity. Another example of a disease which may be treated with a composition of the present invention may be asthma which recent research indicates may be caused by a bacterial infection. The composition of the present invention may further be used to remove oral plaque as this is also caused by a bacterial biofilm.

In another embodiment the composition may be applied to infections wherein the infection comprising a microbial biofilm is in a patient with inserted foreign bodies, such as hip, knee, heart valve replacements or internal or external catheters. Such patient may have a compromised immune system either due to an existing illness, surgery or due to immunosuppressive medication.

In one embodiment of the present invention the composition is administered to the skin or a wound of a mammal using a patch, bandage, dressing or any other carrier device applicable to the skin. For example an exchangeable dressing comprising the composition of the present invention may be used. Alternatively a standard dressing or bandage incorporating a sponge comprising the composition of the present invention may be applied. Additionally, a dressing incorporating a continuous or semi-continuous flow of the composition of the present invention may also be applied.

In another embodiment the composition of the present invention may be used to clean and/or disinfect industrial and medical equipment, especially equipment where a high hygiene standard is necessary. Examples of industries in which it is foreseen that a composition according to the present invention may be used include but are not limited: any food-producing industry, such as the dairy industry, the meat, poultry or aquaculture industry or the brewery industry. Examples of devices which may be treated with a composition of the present invention include but are not limited to medical or industrial devices, such as surgical instruments, catheters or pipelines.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Material and Methods

### Bacterial Strains

The wild-type P. aeruginosa PAO1 used for the planktonic and biofilm experiments was obtained from the Pseudomonas Genetic Stock Center (www.pseudomonas.med.ecu.edu, strain PAO0001). The wild-type S. aureus 8325-4, used for planktonic and biofilm experiments was described by Novick, R. P. 1967.

The clinical isolates of P. aeruginosa were obtained from chronically infected cystic fibrosis patients at the University Hospital of Copenhagen.

### Growth Media

For plating, Luria broth (LB) medium mix with 2.0% agar was used. For all experiments including bacterial biofilms, AB minimal medium supplemented with glucose was used except if different is mentioned. AB minimal medium consists of: A standard buffer system consisting of (NH₄)₂SO₄ (15.1 mM), Na₂HPO₄.2H₂O (33.7 mM) and KH₂PO₄ (22.0 mM. NaCl (0.051 M), MgCl₂ (1 mM), CaCl₂ (0.1 mM), and trace metals (100 μl/liter). The trace metal solution contained CaSO₄.2H₂O (200 mg/liter), FeSO₄.7H₂O (200 mg/liter), MnSO₄.H₂O (20 mg/liter), CuSO₄.5H₂O (20 mg/liter), ZnSO₄.7H₂O (20 mg/liter), CoSO₄.7H₂O (10 mg/liter), NaMoO₄.H₂O, and H₃BO₃ (5 mg/liter). Furthermore, experiments where the standard buffer system of the AB minimal medium was replaced by sodium acetate were performed in all the below examples. The exchange of buffer did not affect the results.

### Growth of Bacteria

Two types of biofilm setups were used, a continuous flow system and a static system:

The continuous flow system is based on once through flow chambers perfused with sterile AB minimal medium containing 0.3 mM glucose as described by Christensen et al. (1999).

The static biofilm setup is based on biofilms growing in microtiter dishes with AB minimal medium containing 0.3 mM glucose as described by O'toole et al (1999). Planktonic cultures were grown in shake flasks at 37° C.

### Antimicrobial Treatments

Continuous flow biofilm tolerance to acetic acid was assessed by growing P. aeruginosa or S. aureus biofilms for three days, then subsequently at day three to four supplementing the AB minimal medium with different concentrations of acetic acid or HCl (HCL served as a as control to acetic acid treatments).

Static biofilm tolerance to acetic acid was assessed by exchanging the AB minimal medium of 24 h old biofilms with AB minimal medium supplemented with different concentrations of acetic acid or HCl as control. To raise the pH of either acetic acid or HCl, NaOH was added in different concentrations.

### Example 1

The efficacy of acetic acid with respect to eradication of mature biofilms was tested by treating a 3-day-old flow chamber biofilm of either P. aeruginosa or S. aureus with 0.5% and 1.0% acetic acid for 24 hours. Due to the buffer capacity of the AB minimal medium used, the pH of the solution became 4.33. As control similar biofilms were treated with AB minimal medium adjusted to pH 4.33 using HCl. After the treatment the biofilm biomasses were harvested mechanically from the flow chambers and plated on LB plates for determination of viability. Treatment with either 0.5 or 1.0% acetic acid completely eradicated P. aeruginosa biofilms, whereas the HCl treatment had no effect. As for S. aureus treatment with 0.5% acetic acid reduced the number of viable cells, whereas complete eradication was obtained using 1.0% acetic acid.

### Example 2

To verify whether the killing capacity of the treatment in example 1 was due to acetic acid alone and not a combination of the constituents of the medium, the experiment in example 1 was repeated using 0.5% or 1.0% acetic in sterile miliQ water in contrast to AB minimal medium supplemented with glucose. Complete eradication was observed when harvesting 0.5% acetic acid treated P. aeruginosa and 1.0% acetic acid treated S. aureus biofilms, compared to the controls.

### Example 3

The kinetics of antimicrobial activity against mature biofilms was tested by treating a 3-day-old continuous flow chamber biofilms of either P. aeruginosa with 0.5% or S. aureus with 1.0% acetic acid for 24 hours. Due to the buffer capacity of the AB minimal medium used the pH of the solution became 4.33. As control similar biofilms were treated with AB minimal medium adjusted to pH 4.33 by addition of HCl. After the treatment the biofilms were harvested mechanically, by scraping with a sterile scalpel, from the flow chambers and plated on LB plates for determination of viable counts. Complete killing of all bacteria was reached after 3 hours using 0.5% acetic acid against P. aeruginosa and 1.0% against S. aureus.

### Example 4

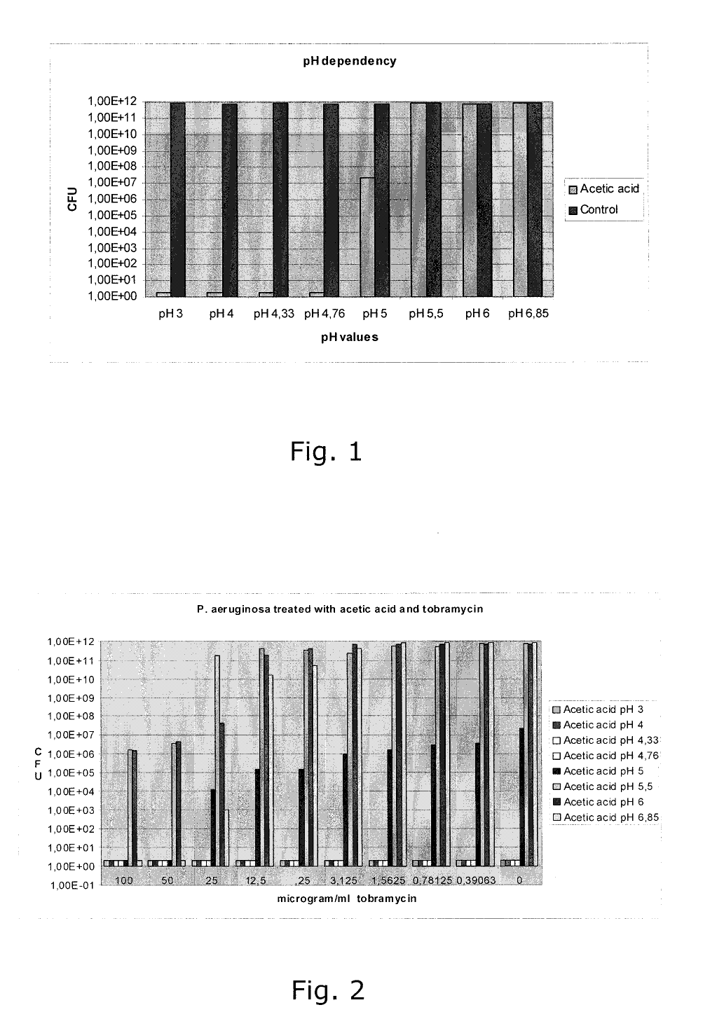

To elucidate the pH dependency of the antimicrobial effect of acetic acid static 24 hour old biofilms were treated with a selection of 0.5% acetic acid solutions with increasing pH. Acetic acid in the AB minimal medium resulted in a pH value of 4.33, lower pH values were obtained by addition of HCl whereas higher pH values by addition of NaOH. This was compared to AB minimal media adjusted to the same range of pH by HCl or NaOH alone. As seen from FIG. 1, acetic acid eradicated biofilms of P. aeruginosa in the pH interval 3-4.76. At pH 5 a reduced effect is observed whereas at pH 5.5 and above no effect of acetic acid is observed. This pH dependency is due to the dissociation of acetic acid, which is at equilibrium at pH 4.76 (pKa value). Below pH 4.76 the equilibrium is shifted to the left i.e. acetic acid, and above pH 4.76 the equilibrium is shifted to the right i.e. the corresponding base.

CH₃COOH→CH₃COO⁻+H^{+pH>4.76}

CH₃COOH←CH₃COO⁻−H^{+pH<4.76}

CH₃COOH═CH₃COO⁻+H^{+pH=4.76}

The results of the present experimental scenario demonstrate that it is not an acidic effect (low pH values) per se, which is the cause of the kill; it is the acetic acid molecule itself.

### Example 5

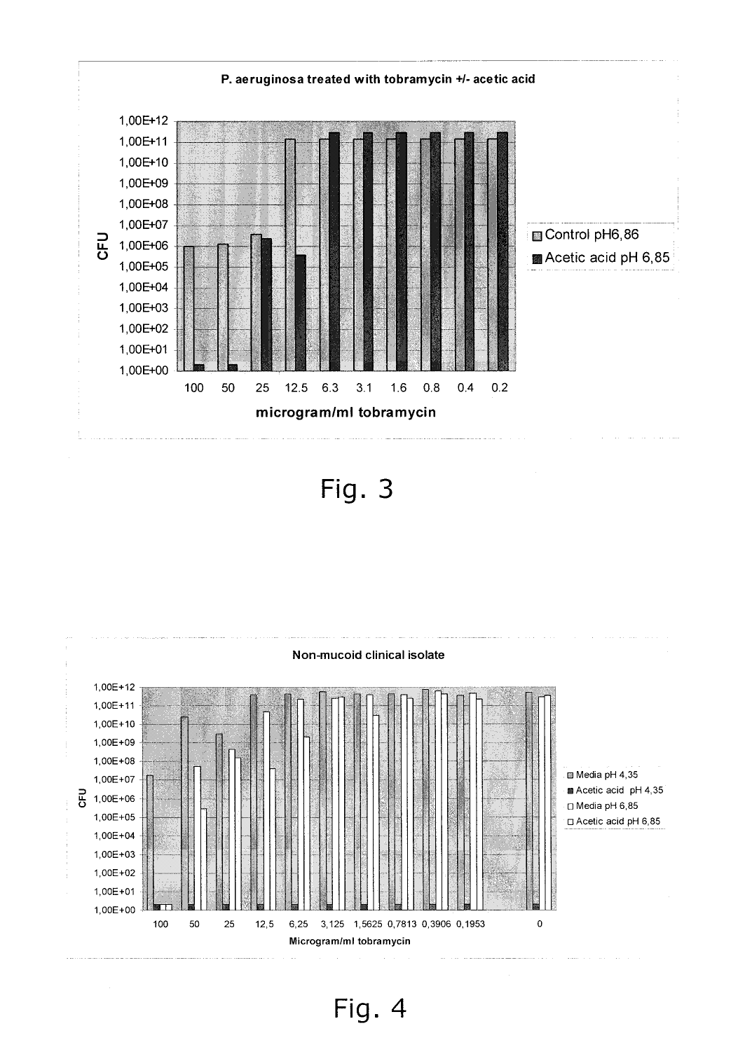

To elucidate a synergistic antimicrobial effect of acetic acid and antibiotics, static biofilms of P. aeruginosa were grown for 24 hours prior to subsequent treatment with increasing concentrations of tobramycin and 0.5% acetic acid in the pH interval 3-6.85. The pH was adjusted by supplementing the AB minimal medium with either HCl or NaOH. When comparing acetic acid at pH 6.85 with and without tobramycin (FIG. 1, 2) a synergistic, antimicrobial effect exists between acetic acid and antibiotics. With pH values above 4.76, (FIG. 1) which is the dissociation point for acetic acid, the effect of acetic acid alone is decreasing. Acetic acid alone at pH 6.85 has no antibacterial effect on the bacteria (FIG. 1) however in the presence of tobramycin an effect on viable counts was observed including at pH 6.85 (FIG. 2). Tobramycin alone reduced the bacterial viability 5 fold at concentrations above 12.5 μg/ml (see FIG. 3). Acetic acid combined with tobramycin reduced the bacterial viability at pH values above 4.76 and showed a greater effect on viability than tobramycin alone (FIG. 3).

### Example 6

To evaluate the clinical antimicrobial potential of acetic acid, static biofilms of clinical P. aeruginosa isolates were treated with acetic acid as described for example 5. As seen from FIG. 4, acetic acid eradicates the biofilms at a pH value below the dissociation point (pH 4.76) of acetic acid. Additionally the same synergistic effect between acetic acid and antibiotics is seen as for example 5.

### Example 7

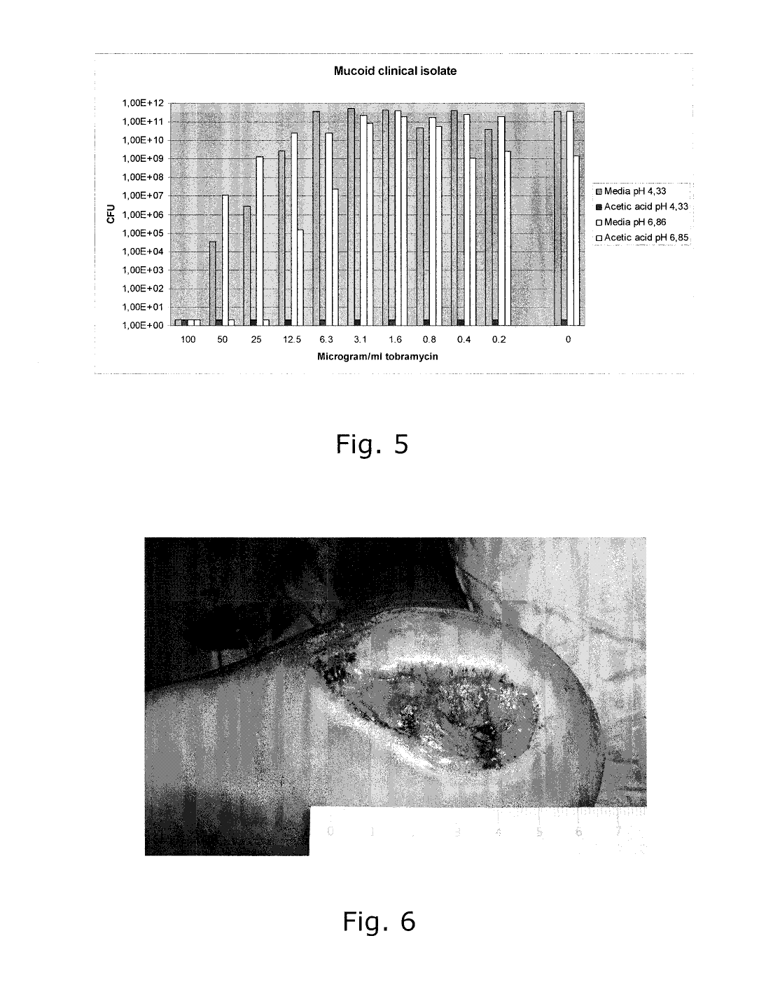

To evaluate the clinical potential of acetic acid towards the clinical important mucoid (alginate over-producing) P. aeruginosa phenotype, static biofilms of clinical isolated mucoid P. aeruginosa were treated with acetic acid as described for example 5. As seen from FIG. 5, acetic acid eradicates the biofilms at a pH value below the dissociation point (pH 4.76) of acetic acid. Additionally the same synergistic effect between acetic acid and antibiotics is seen as for example 5.

### Example 8

To further evaluate the clinical potential of buffered 0.5% acetic acid solution towards the clinical important mucoid (alginate over-producing) P. aeruginosa phenotype, it was tested in the treatment of a chronic heel ulcer.

### Medical History:

A 38 year old male with type 2 mellitus diabetes associated neuropathy was presented to a wound healing clinic. A heel ulcer was obtained during a vacation due to strenuous walking. The 38 year old male had the following prior history of treatment with no apparent improvement in wound healing (over period of three months):

Off-loading, therapeutic shoes and aircast.

Wound treatment with silver dressings and compression.

Several courses of antibiotics.

### Treatment:

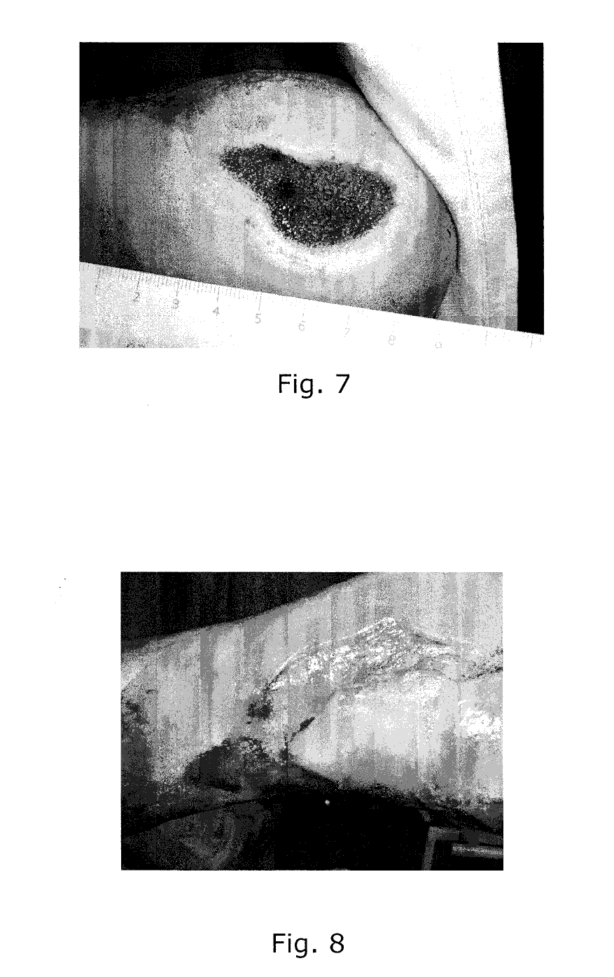

Treatment of the chronic wound (FIG. 6 [day 0]) with phosphate buffered 0.5% acetic acid (patient continued antibiotic therapy) was performed 6×20 minutes per day, for 11 days (continuous). On day 11 the infection had been eradicated and wound healing had begun as shown in FIG. 7 (day 11).

### Example 9

To further evaluate the clinical potential of buffered acetic acid solution towards the clinical important mucoid (alginate over-producing) P. aeruginosa phenotype, it was tested in the treatment of a chronic leg ulcer.

### Medical History:

A 73 years old female with type 2 mellitus diabetes, and a 3 year long history of leg ulcers. The patient have received an ulcer debridement and split skin transplant in 2006 with only temporarily success as the ulcer reoccurred after 3 months. The patient was considered unfit for a new operation de to her heart condition. Cultures prior to treatment have shown Pseudomonas aeruginosa and Staphylococcus aureus. The patient received anti-Staphylococcus treatment due to infected toe on the contralatteral leg (Heracillin).

### Treatment:

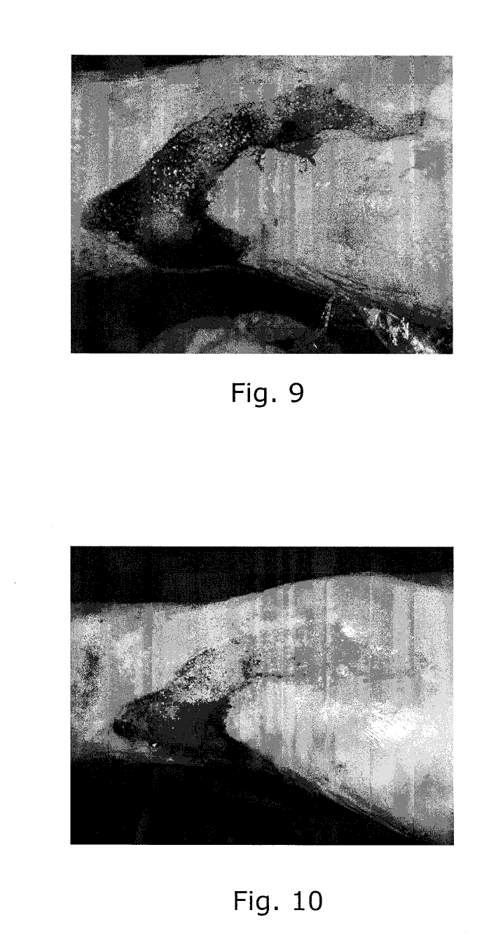

Treatment of wound (FIG. 8 [day 0]) with phosphate buffered acetic acid (patient continued antibiotic therapy) was performed 6×20 minutes per day, for 6 days (continuous). After 3 days a significant improvement in the wound healing process was evident as shown in FIG. 9 (day 3) and after 6 days the infection was eradicated and the wound healing process was proceeding well as shown in FIG. 10 (day 6).

### REFERENCES

Novick, R. P. 1967. Properties of a cryptic high-frequency transducing phage in Staphylococcus aureus. Virology 33:155-166.
Christensen, B. B., Sternberg, C., Andersen, J. B., Palmer, R. J., Jr, Nielsen, A. T., Givskov, M. & Molin, S. (1999). Molecular tools for study of biofilm physiology. Methods Enzymol 310, 20-42.
O'Toole, G. A., Pratt, L. A., Watnick, P. I., Newman, D. K., Weaver, V. B. and Kolter, R. (1999) Genetic approaches to study of biofilms. Methods Enzymol 310, 91-109.

1. A composition comprising:
a) a 0.01-20% wt/wt acetic acid; and
b) a physiologically tolerable buffer capable of maintaining acetic acid at a pH in the range of 2-7.

2-17. (canceled)

18. The composition according to claim 1, wherein said composition further comprises an antibiotic.

19. The composition according to claim 18, wherein said antibiotic is selected from the group consisting of: amino glycosides, macrolides, fluoroquinolones, ceftazidimes, tetracyclines, sulfonamides, beta-lactams and antimicrobial peptides.

20. The composition according to claim 1, wherein the buffer is selected from the group consisting of: sodium acetate, sodium bicarbonate, phosphate buffers, tris buffers and HEPES buffer.

21. The composition according to claim 1, wherein said composition further comprises one or more compounds selected from the group consisting of: detergents, anti-microbial agents and disinfectants.

22. A method of inhibiting the presence of a microbial biofilm on a surface comprising:
contacting a surface that may comprise a microbial biofilm with a composition comprising 0.01-20% wt/wt acetic acid.

23. The method of claim 22, wherein said surface is one of a medical or industrial device.

24. A method of reducing bacterial growth comprising contacting the composition according to claim 1 to a microbe.

25. A method of inhibiting an infection comprising a microbial biofilm comprising contacting the composition according to claim 1 to said infection.

26. The method according to claim 25, wherein the infection comprising a microbial biofilm is in a patient with reduced immune response.

27. The method according to claim 26, wherein the reduced immune response is caused by cystic fibrosis, diabetes, age or obesity.

28. The method according to claim 25, wherein the infection comprising a microbial biofilm is in a patient with an implanted hip, knee, heart valve, or internal or external catheter.

29. The method according to claim 25, wherein the infection comprising a microbial biofilm is present in the lungs, in a chronic wound, on or around an implant on or around a prosthetic device, in or around catheters, or in or around the oral cavity.

30. The method according to claim 25, wherein the composition is administered to the skin or a wound of a mammal using a patch, bandage, dressing or any other carrier device applicable to the skin.
